(19) United States
(12) Patent Application Publication
(10) Pub. No.: US 2026/0224812 A1
(43) Pub. Date: Aug. 6, 2026

(54) Fluid Bolus Recommendation

(71) Applicant: Becton, Dickinson and Company, Franklin Lakes, NJ (US)

(72) Inventors: Brennan Michael Schneider, Irvine, CA (US); Sai Prasad Buddi, Santa Ana, CA (US); Brian R. Hipszer, Irvine, CA (US); Zhongping Jian, Irvine, CA (US); Pamelpreet Kaur Kang, Irvine, CA (US)

(21) Appl. No.: 19/155,112

(22) PCT Filed: Aug. 26, 2024
(86) PCT No.: PCT/US2024/043866

§ 371 Date: Aug. 8, 2025

Related U.S. Application Data
(60) Provisional application No. 63/579,793, filed on Aug. 30, 2023.

(51) Int. Cl. A61M 5/172; G16H 20/17; G16H 40/67

(52) U.S. Cl. CPC A61M 5/1723; G16H 20/17; G16H 40/67; A61M 2230/30

(57) ABSTRACT

A method for managing fluid administration to a patient comprises accessing a plurality of features associated with administration of a fluid bolus to a patient, receiving a first fluid bolus recommendation indicating a predicted change in a physiological parameter of the patient in response to the fluid bolus based on the plurality of features, determining a second fluid bolus recommendation based on the first fluid bolus recommendation and a subset of the plurality of features, and providing the second fluid bolus recommendation.

### RELATED APPLICATION

This application claims priority to U.S. Provisional Application No. 63/579,793, filed Aug. 30, 2023, and entitled FLUID BOLUS RECOMMENDATION, the disclosure of which is hereby incorporated by reference in its entirety.

### BACKGROUND

The present disclosure generally relates to the field of fluid administration, including devices and methods for hemodynamic management capable of facilitating fluid administration, transfusion of blood products, and administration of blood pressure supporting medications.

### SUMMARY

Described herein are devices, methods, and systems relating to management of fluid bolus administration to patients.

For purposes of summarizing the disclosure, certain aspects, advantages and novel features have been described. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

Any of the example methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loudspeakers, headphones, pressure transducers, temperature transducers, or using any combination of suitable technologies.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements.

FIG. 1 illustrates an example system for administering IV fluid and/or medication to a patient in accordance with one or more examples.

FIG. 2 illustrates a system for delivery of one or more fluids to a patient using a control device (e.g., the control device of FIG. 1) in accordance with one or more examples.

FIG. 3 illustrates a data flow for data inputs to a bolus recommendation engine in accordance with one or more examples.

FIG. 4 provides a flowchart illustrating a process for providing a fluid bolus recommendation in accordance with one or more examples.

FIG. 5 illustrates a flow diagram for analysis and/or selection of features from a candidate feature set in accordance with or more examples.

FIG. 6 provides a flowchart illustrating a process for providing a fluid bolus recommendation in accordance with one or more examples.

FIG. 7 illustrates an example structure of a responsiveness estimation engine in accordance with one or more examples.

FIG. 8 illustrates a responsiveness framework for predicting patient response to fluid boluses in accordance with one or more examples.

### DETAILED DESCRIPTION

The headings provided herein are for convenience only and do not necessarily affect the scope or meaning of the claimed invention.

Although certain preferred examples are disclosed below, it should be understood that the inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

Certain reference numbers are re-used across different figures of the figure set of the present disclosure as a matter of convenience for devices, components, systems, features, and/or modules having features that may be similar in one or more respects. However, with respect to any of the examples disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Where an alphanumeric reference identifier is used that comprises a numeric portion and an alphabetic portion (e.g., ‘10a,’ ‘10’ is the numeric portion and ‘a’ is the alphabetic portion), references in the written description to only the numeric portion (e.g., ‘10’) may refer to any feature identified in the figures using such numeric portion (e.g., ‘10a,’ ‘10b,’ ‘10c,’ etc.), even where such features are identified with reference identifiers that concatenate the numeric portion thereof with one or more alphabetic characters (e.g., ‘a,’ ‘b,’ ‘c,’ etc.). That is, a reference in the present written description to a feature ‘10’ may be understood to refer to either an identified feature ‘10a’ in a particular figure of the present disclosure or to an identifier ‘10’ or ‘10b’ in the same figure or another figure, as an example.

Certain standard anatomical terms of location are used herein to refer to the anatomy of animals, and namely humans, with respect to various examples. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa. It should be understood that spatially relative terms, including those listed above, may be understood relative to a respective illustrated orientation of a referenced figure.

### Fluid Management Systems

Excessive and/or inadequate fluid administration can cause complications. Goal-directed fluid management can help optimize the amount and/or timing of fluid administration. Some examples described herein relate to guiding effective intravenous fluid administration during surgery and/or otherwise.

Maintaining adequate oxygen delivery during surgery can prevent damage to vital organs and resultant complications. Maintaining adequate intraoperative cardiac output is vital to oxygen delivery. Hemodynamic-guided fluid management, also called goal-directed therapy, can help optimize cardiac output and/or may improve outcomes in high-risk surgical patients.

Goal-directed therapy can require clinicians to follow standardized systems (e.g., computer systems) that determine when fluids should be given. A common feature of some systems is an effort to maintain a predefined stroke volume (SV) and limit SV variation (e.g., less than 12%). The complexity and variety of goal-directed therapy systems can make them challenging to implement. Accordingly, adherence to such systems is often poor.

Using invasive (e.g., arterial) pressure information, example systems can recommend a fluid administration when patients are likely to respond to fluid bolus with a predefined increase in SV. Automated assessment of hemodynamic status and prompting specific fluid recommendations may facilitate intraoperative fluid management during surgery.

Some example systems utilize an open-loop fluid management workflow. Systems can perform automatically and/or clinicians may be guided by the systems while retaining full control of fluid administration. Fluid management systems can perform various functions, including (1) integrating monitored hemodynamic variables and/or continuously analyzing patients' fluid responsiveness; (2) analyzing the response to fluid boluses; and/or (3) predicting patients' current fluid responsiveness and, when appropriate, prompting clinicians to consider a fluid bolus.

Some example systems and/or methods provided involve hemodynamic monitoring including dynamic parameters of fluid responsiveness (e.g., fluid predictors) derived from various physical data, which can include arterial pressure waveforms among other things.

The systems described herein can utilize, among other physiologic data, dynamic predictors of fluid responsiveness and/or other dynamic data. Dynamic predictors can include pulse-pressure variation (PPV), stroke volume variation (SVV), plethysmograph variability, and/or electrocardiogram (EKG) waveform characteristics, the description will simply refer to the group as the “Fluid Predictors” or FP. This term should be taken to mean any of the described predictors of fluid responsiveness.

Other terms and abbreviations used herein include:
CO—Cardiac output;
APCO—Arterial pressure cardiac output;
SWI—Stroke Work Index;
CI—Cardiac Index;
dP/dt—maximal rate of change of an arterial blood pressure waveform;
Patient or Subject—the “patient” or “subject” is the organism being monitored by and managed by the system (in one example, the patient may be a human being; however, patients can include animals);
Vital Signs or Vitals—Any statistical measure of a physiologic process taking place in a patient—including waveforms derived from physiologic processes. Vitals can include, for example: Heart Rate (HR)—the number of ventricular contractions per minute; Stroke Volume (SV)—the volume of blood ejected by the left ventricle during contraction in milliliters; Systolic Blood Pressure (SBP)—the highest blood pressure felt in the systemic arterial vascular tree during a cardiac cycle; Diastolic Blood Pressure (DBP)—the lowest blood pressure felt in the systemic arterial vascular tree during a cardiac cycle; Mean Arterial Pressure (MAP)—the average blood pressure in the systemic arterial system over one or more cardiac cycles, typically calculated as ((SBP+DBP+DBP)/3); Systemic Vascular Resistance (SVR)—an amount of force exerted on circulating blood by the vasculature of the body; Cardiac Output—the total volume of blood ejected by the left ventricle over one minute;
Dynamic Predictor (DP)—one or more measures of preload dependence derived from an arterial pressure waveform, plethysmograph waveform, EKG waveform, thoracic ultrasound, bioimpedance, bioreactance, and including specific maneuvers such as passive leg raising and tele-expiratory pause;
Intravenous fluid (IV Fluid)—any fluid intended for administration intravenously to a monitored subject for the purpose of intravascular volume expansion or replacement, or acting as a carrier for intravenous medications. IV Fluid would therefore include, but not be limited to: crystalloid solutions like Lactated Ringer's Solution, Normal Saline, Dextrose Solutions, Plasmalyte, and in general balanced salt solutions and sugar solutions; colloidal solutions like albumins, starches, and similar; and blood products and blood analogs like whole blood, platelets, fresh frozen plasma, cryoprecipitate, packed red blood cells, salvaged cellular solutions, or any substitutes meant to mimic or replace these products;
Fluid bolus—an administration of a specific volume of IV Fluid over a discrete timespan;
“Efficacy” of a Fluid Bolus—the degree to which the intravascular administration of said fluid increases the cardiac output; or the degree to which the intravascular administration of said fluid improves the delivery of oxygen to the tissues, for example;
Prediction—the calculated percent increase in cardiac output that a fluid bolus would be expected to cause in the patient;
Vasoactive Medications—medications controlled by the system that could include those intended to manipulate blood pressure and cardiac output such as, for example, ephedrine, phenylephrine, norepinephrine, epinephrine (adrenaline), dopamine dobutamine, milrinone, dopexamine, nitroglycerine, nitroprusside, and other vasopressors, inotropes, and vasodilators.

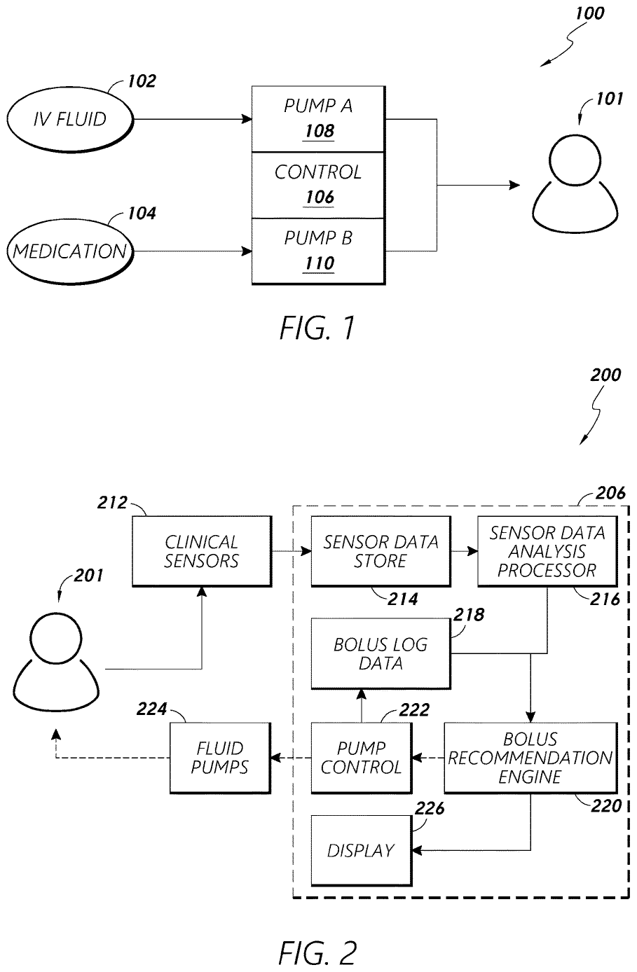

FIG. 1 illustrates an example system 100 for administering IV fluid 102 and/or medication 104 to a patient 101 in accordance with one or more examples. The system 100 may comprise a control device 106 (e.g., control circuitry) configured to manage hemodynamic delivery of the IV fluid 102 (e.g., IV fluid bags) and/or medication 104 to the patient 101 via a first pump 108 (e.g., fluid pump and/or infusion pump), a second pump 110 (e.g., fluid pump and/or infusion pump), and/or various additional pumps. The control device 106 may be electronically coupled to the first pump 108 and/or second pump 110 via various circuitry and/or electronic interfaces.

The control device 106 can include one or more sensors and/or display devices. For example, display devices can include a display screen and/or user interface devices (e.g., keyboard, mouse, etc.). The first pump 108 may be coupled to the IV fluid 102 and/or the second pump 110 may be coupled to the medication 104, which can include various containers containing fluid medications. The IV fluid 102 and/or medications 104 can be fluidically coupled to the patient 101 via tubing and/or administration of the IV fluid 102 and/or medication 104 can be administered to the patient 101 and/or dynamically controlled by the control device 106.

The control device 106 can include one or more processing units and/or one or more computer readable storage medium. The processing units may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof. The computer readable storage medium may include one or more memories for storing data, including read only memory (ROM), random access memory (RAM), magnetic RAM, core memory, magnetic disk storage mediums, optical storage mediums, flash memory devices and/or other machine-readable mediums for storing information. The computer readable storage medium may be embodied in one or more portable or fixed storage devices, optical storage devices, wireless channels, and/or various other storage mediums capable of storing that contain or carry instruction(s) and/or data.

The control device 106 can be coupled to the fluid pumps via one or more interface ports. The interface ports can include one or more of a standard USB port and a standard serial port. The USB connector can also be used to import patient data in real-time from another source such as a patient monitor. Optionally, an external component may be connected to the control device 106 to allow for direct monitoring of patient vital signs by the control device 106. One or more interfaces can allow data to be transferred to and from the control device 106 for sharing data with other networked equipment.

The first pump 108, second pump 110, and/or control device 106 can comprise a single device and/or may be integrated in a single housing. Alternatively, the first pump 108 and/or second pump 110 can be external to the control device 106. The first pump 108 can be configured to regulate and/or drive the flow of IV fluid 102 to the patient 101 and/or to select which fluid to use from one or more fluid containers. The IV fluid(s) can include one or more of crystalloids, colloids, or blood products as well as other fluids. The second pump 110 can be integrated with or external to the control device 106 and/or can include syringe pump systems for use with multiple medication vials and/or containers.

Various tubing can be coupled with the control device 106 after flushing and/or prior to use. In some examples, multiple IV tubes can be connected to the patient 101.

One end of the IV tubing can include bag taps for use with IV solutions, colloids, and blood products. The opposite end of the IV tubing can be a male luer lock for connection to IV tubing sets and/or claves. The IV tubing can also have side ports distal to the main fluid pump which the medication syringes from the medication vials can be attached to.

FIG. 2 illustrates a system 200 for delivery of one or more fluids to a patient 201 using a control device 206 (e.g., the control device 106 of FIG. 1) in accordance with one or more examples.

In some examples, a patient 201 may be monitored through use of one or more clinical sensors 212 and/or monitors. The various sensors 212 may be coupled to the control device 206 via various circuitry. The sensors 212 can be integrated with and/or may be separate from (e.g., detached and/or remotely connected to) the control device 206. The sensors 212 may be configured to measure vitals and/or various physiological data from the patient. Such measured data may be stored at a sensor data store 214 of the control device 206. In some examples, measured data may be filtered for noise and/or validity prior to storage at the data store 214. The sensor data store 214 may be configured to store additional data, including population data and/or various data collected from patients other than the patient 201.

Data from the sensor data store 214 may be accessed by a sensor data analysis processor 216. The processor 216 may be configured to make predictions regarding the likely efficacy of a fluid bolus. In some examples, the processor 216 may be configured to analyze and/or compare sensor data from the sensors 212 to data obtained from a previous population of patients (e.g., patients having similar vitals) in response to the fluid bolus.

Data from the processor 216 may be transmitted automatically and/or on request to a bolus log data store 218 and/or a bolus recommendation engine 220. In some examples, data may be sent to the log data store 218 when a fluid bolus is initiated and/or terminated. The log data store 218 may also receive inputs from a pump controller 222 (e.g., when fluid boluses are initiated or terminated) including certain details of the bolus being administered.

The bolus recommendation engine 220 may be configured to analyze historical data of the patient 201 and/or to predict a current efficacy of a fluid bolus. Such prediction can involve analysis of sensor data from the clinical sensors 212 and/or sub-analyses.

The engine 220 may be configured to determine a combined prediction in cardiac output for a current state of the patient 201 based on log data 218, sensor data 214, and/or various population data. The combined prediction may optionally cause a transmission of a command from the engine 220 to the pump controller 222 to allow the pump controller 222 to automatically cause activation and/or deactivation of one or more fluid pumps 224 configured to pump one or more fluids to the patient 201. However, the engine 220 may additionally or alternatively present a prediction and/or recommendation to a display 226 (e.g., monitor) to allow a clinician to view the results and/or manually pump and/or command pumping of the fluid pumps 224.

The fluid pump(s) 224 may be externally controlled fluid pumps which contain an independent command interface, alarm system, and/or configuration. The control of the fluid pumps 224 can be achieved over serial, network, wireless, Bluetooth, and/or other electronic protocols. The fluid pumps 224 can comprise one, two, or more than two physical fluid pumps 224.

In alternative examples, the fluid pump(s) 224 may be integrated components of the control device 206. The fluid pumps 224 may or may not include alarm and/or control configurations. If the fluid pumps 224 do not include alarms and controls, the alarms and controls for the pumps 224 can be included in the control device 206. A user interface (e.g., the display 226 and/or associated devices) of the control device 206 can be used to affect some aspects of the fluid pumps 224.

The pumps 224 may be configured to deliver IV fluid and/or medications to the patient 201 at determined rates and/or times and/or as controlled by a clinician. The display 226 can comprise a touch-screen interface for monitoring vital signs of the patient and/or for entering patient data and user preferences into the control device 206.

The control device 206 can be configured to monitor and/or store various hemodynamic data including variables such as heart rate, mean arterial pressure, and estimated advanced variables using pulse contour analysis including SV, SV variation, and systematic vascular resistance. Data can be recorded at a start and/or end time of each bolus, along with the fluid type and volume. The engine 220 may be configured to utilize fluid delivery data and/or hemodynamic data to estimate the percent change in SV (e.g., change in SV) resulting from a fluid bolus. Whenever fluid is given within prescribed limits, the engine 220 may be configured to calculate the expected change in SV by overlaying the start time and stop time of the fluid bolus on top of the SV measurements and/or displaying such data at the display 226.

Predictions at the engine 220 of the patient's 201 current fluid responsiveness can combine predictions from a population model and a bolus log model. The population model describes the relationship between SV variation and predicted change in SV. The bolus log model uses the hemodynamic responses to past fluid boluses to determine whether a patient is fluid responsive. The engine 220 can generate a predicted change in SV by identifying boluses that were given in a similar hemodynamic state and aggregating those responses. In some examples, the engine 220 may be configured to compare the measured change in SV to the predicted change in SV for those boluses being used in the bolus log model and can correct the prediction model for systematic biases (e.g., the model is either overestimating or underestimating the patient's response to fluid). In some examples, the average of the population model prediction and the bolus log prediction can be weighted at the engine 220 by the quality of the information in the bolus log model to produce a final prediction. The engine 220 can determine whether a fluid bolus suggestion should be generated. If the predicted change in SV is greater than a threshold setting, the output of the engine 220 may be a fluid suggestion prompt that is displayed on the display 226.

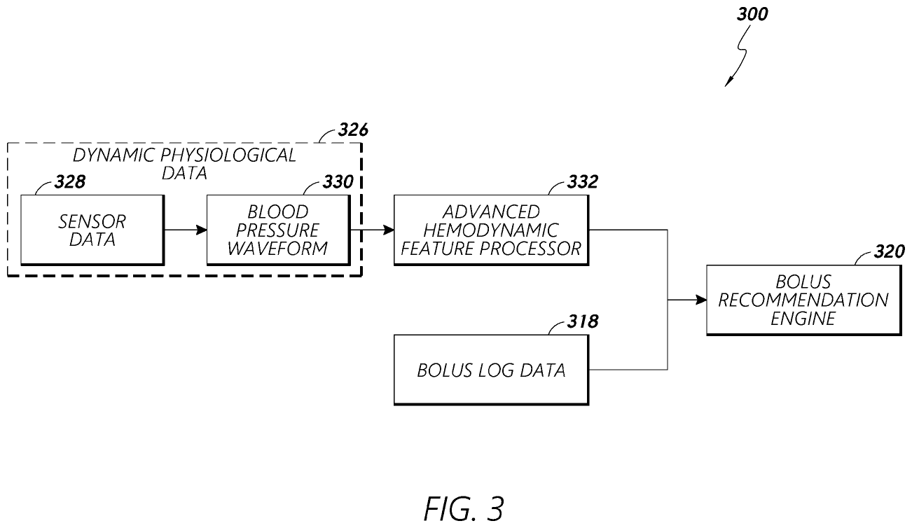

FIG. 3 illustrates a data flow 300 for data inputs to a bolus recommendation engine 320 in accordance with one or more examples. In some examples, various dynamic physiological data 326 can be collected from various clinical sensors and/or monitors. For example, physiological data 326 can include sensor data 328 related to a monitored patient and/or collected from one or more sensors and/or monitors. Physiological data 326 can also include a blood pressure waveform 330 for the patient. In some examples, the blood pressure waveform 330 can be generated and/or derived using the sensor data 328. For example, the blood pressure waveform 330 can present measured sensor data 328 tracked over a period of time.

The dynamic physiological data 326 can be input to an advanced hemodynamic feature processor 332. The processor 332 can be configured to perform an analysis and/or sub-analysis on the physiological data 326 to generate additional features (e.g., SV, SV rate of change, APCO, etc.) related to the patient. Example features generated by the processor 332 are described in further detail herein. Features generated by the processor 332 can be transmitted to the bolus recommendation engine 320 for generating a prediction of patient response to a bolus and/or a recommendation for providing a bolus to the patient. The processor 332 may additionally or alternatively receive demographic data and/or data relating to previous patients and/or patients having similar traits to a current patient. The demographic data can include sensor data and/or blood pressure waveform data received from the previous patients.

In some examples, the engine 320 may additionally receive bolus log data 318 relating to boluses previously provided to the patient and/or to similar patients. The data 318 can include bolus start times, bolus stop times, and/or volumes of boluses previously provided to the patient and/or other patients. The data 318 can instruct the engine 320 as to the patients' responses to past boluses to assist in generating recommendations for future boluses. In some examples, data 318 can include intervention data and/or any data which may be used in determining interventive actions, which can include bolus log data.

Predictions and/or recommendations generated by the engine 320 may be presented in a display device for viewing by a clinician. In some examples, the display device can provide an interface for initiating actions of one or more fluid pumps and/or may present options for selection by the clinician. For example, where a fluid bolus is recommended, the display device may indicate that a bolus is recommended and/or may be present a “start bolus” and/or “decline” option, each of which may be selectable by the clinician.

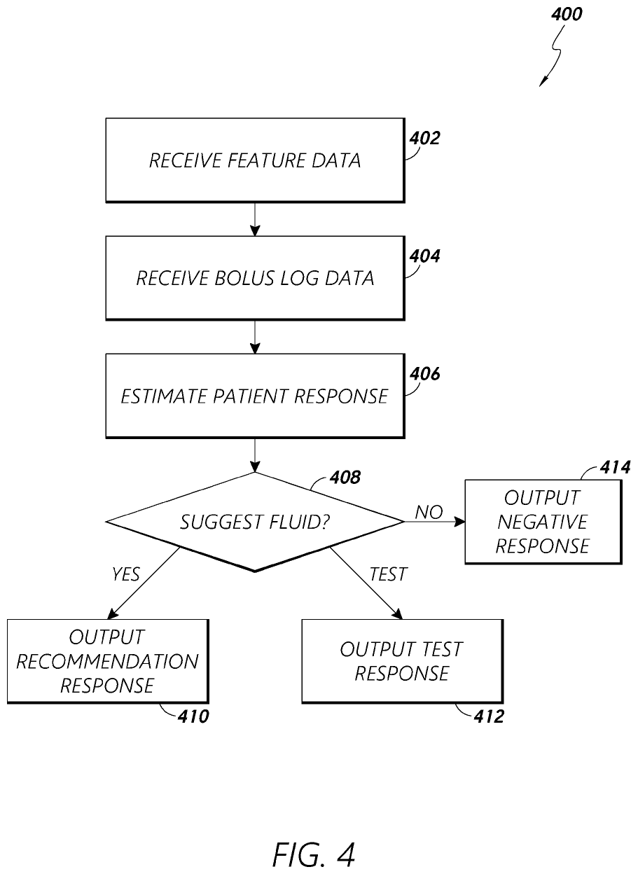

FIG. 4 provides a flowchart illustrating a process 400 for providing a fluid bolus recommendation in accordance with one or more examples. The process 400 may involve an automatic provision of a fluid bolus in response to a recommendation and/or may involve display of an output for viewing and/or response by a clinician.

At a step 402, the process 400 involves receiving feature data from a processor. The processor may be configured to generate the feature data using various dynamic physiological data specific to a single patient and/or multiple patients. Example feature data can include basic features (e.g., cardiac output, stroke volume, and/or amplitudes, areas, slopes, durations, and/or display parameters related to cardiac output, stroke, volume, and/or other features), interaction features (e.g., baroreflex sensitivity), complexity features (e.g., sample entropy, approximate entropy, variability), and/or spectral features (e.g., spectral power at different frequency bands). Features can be specific to a point along a waveform. For example, each feature may be measured at 20-second intervals. In some examples, features can include rate of change values for one or more features measured across a period of time. For example, a feature can comprise a change of stroke volume across a 20-second period.

The feature data may be transmitted by a processor and/or may be configured to be received at a bolus recommendation engine and/or similar device.

At a step 404, the process 400 involves receiving bolus log data. Bolus log data may be accessed from a log data store of a control device. In some examples, bolus log data may be provided to the data store from a pump controller and/or a sensor data analysis processor. Log data can include data related to a specific patient and/or data related to multiple patients.

At a step 406, the process 400 involves estimating a specific patient's response to a potential fluid bolus (e.g., IV fluid and/or medication). Estimating the patient response may involve evaluating and/or comparing the feature data and/or bolus log data.

At decision block 408, the engine may determine whether to suggest fluid based on the estimated patient response. The determined suggestion and/or recommendation may be presented to a clinician for consideration. Where fluid is suggested, a “recommendation” response may be generated at a step 410 and/or a fluid bolus may be provided. Where fluid is not suggested, a negative response may be generated at a step 414 and/or a fluid bolus may not be provided. Where fluid is neither suggested nor not suggested, a “test” response may be generated at a step 412. In response to a “test response,” a limited bolus may be provided and/or suggested.

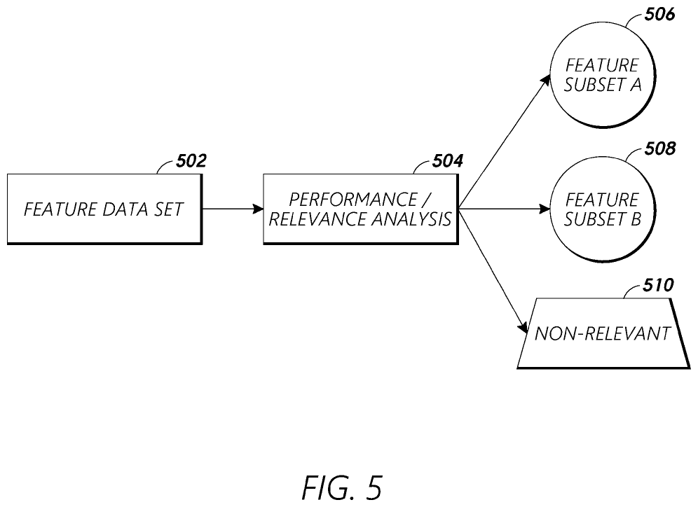

FIG. 5 illustrates a flow diagram for analysis and/or selection of features from a candidate feature set in accordance with or more examples. In some examples, a feature data set 502 may comprise set of various features which may be utilized for generating predictions of a patient's response to a fluid bolus and/or recommendations for administering a fluid bolus to the patient. Example features of the feature data set 502 can include basic features (e.g., cardiac output, stroke volume, and/or amplitudes, areas, slopes, durations, and/or display parameters related to cardiac output, stroke, volume, and/or other features), interaction features (e.g., baroreflex sensitivity), complexity features (e.g., sample entropy, approximate entropy, variability), and/or spectral features (e.g., spectral power at different frequency bands). Features can be specific to a point along a waveform. For example, each feature may be measured at 20-second intervals. In some examples, features can include rate of change values for one or more features measured across a period of time. For example, a feature can comprise a change of stroke volume across a 20-second period. Additional features can include stroke volume index (SVI), SVI rate of change, approximate entropy of diastolic pressure, area from a start of a heartbeat to a systolic maximum with subtracted diastolic pressure, pressure at the dicrotic notch minus a diastolic pressure at an earlier time (e.g., 60 seconds earlier), stroke volume rate of change, rate of change for an approximate entropy of an average pressure at systolic phase, pressure at the dicrotic notch minus diastolic pressure at 0 seconds earlier, skewness approximate entropy rate of change, SWI rate of change, CI rate of change, dP/dt approximate entropy, dP/dt variability rate of change, pulse pressure variation (PPV) rate of change, systemic vascular resistance (SVR), systemic vascular resistance index (SVRI), and/or heart rate approximate entropy.

The feature data set 502 can comprise multiple iterations of a single measurement type measured at different points in time and/or across different periods of time.

For example, the feature data set 502 can comprise cardiac output rate of change measure at a 0-second point, a 20-second point, a 40-second point, and/or a 60-second point. Similarly, the feature data set 502 can comprise stroke volume measure at a 0-second point, a 20-second point, a 40-second point, and/or a 60-second point.

The feature data set 502 may be input to a performance/relevance analysis engine 504 to identify one or more features for inclusion in a first feature subset 506 and/or a second feature subset 508. For example, the engine 504 may be configured to analyze each feature to determine a relevance of the feature for determine a prediction and/or recommendation for administration of a bolus. For example, some features may have a relatively strong correlation with patient response to a fluid bolus and may therefore be considered more relevant than other features for use by a recommendation engine.

In some examples, the relevant features may be sorted into the first subset 506 and/or the second subset 508. Features not sorted into the first subset 506 and/or the second subset 508 may be disregarded and/or sorted into a non-relevant subset 510. The first subset 506 may be associated with a “recommendation” response and/or the second subset 508 may be associated with a “test” response. For example, where the process 400 of FIG. 4 outputs a “recommendation” response, features of the first subset 506 may be applied to a deeper analysis in determining whether to administer a fluid bolus to a patient. Where the process 400 of FIG. 4 outputs a “test” response, features of the second subset 508 may be applied. In some examples, different features may be relatively more relevant and/or useful in deeper analysis for “recommendation” cases than for “test” cases and/or vice versa. Accordingly, the features determined to be most useful for “recommendation” cases may be applied in such cases and/or the features determined to be most useful for “test” cases may be applied such cases. In some examples, features may appear in both the first subset 506 and the second subset 508 and/or in neither.

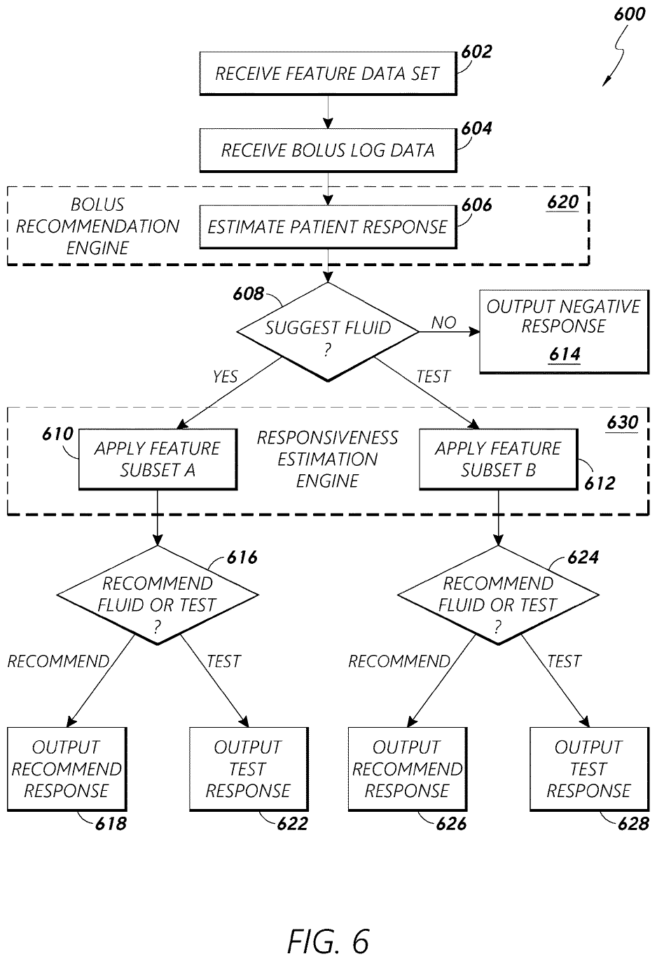

FIG. 6 provides a flowchart illustrating a process 600 for providing a fluid bolus recommendation in accordance with one or more examples. The process 600 may involve an automatic provision of a fluid bolus in response to a recommendation and/or may involve display of an output for viewing and/or response by a clinician.

At a step 602, the process 600 involves receiving feature data from a processor and/or determining (e.g., generating) feature data. The feature data may be associated with administration of a fluid bolus to a patient. The processor may be configured to generate the feature data using various dynamic physiological data specific to a single patient and/or multiple patients. Example feature data can include basic features (e.g., cardiac output, stroke volume, and/or amplitudes, areas, slopes, durations, and/or display parameters related to cardiac output, stroke, volume, and/or other features), interaction features (e.g., baroreflex sensitivity), complexity features (e.g., sample entropy, approximate entropy, variability), and/or spectral features (e.g., spectral power at different frequency bands). Features can be specific to a point along a waveform. For example, each feature may be measured at 20-second intervals. In some examples, features can include rate of change values for one or more features measured across a period of time. For example, a feature can comprise a change of stroke volume across a 20-second period.

The feature data may be transmitted by a processor and/or may be configured to be received at a bolus recommendation engine and/or similar device. In some examples, the feature data may be collected using one or more sensors which may be attached to and/or otherwise in communication with the patient. For example, a sensor can comprise a wrist and/or finger cuff.

In some examples, the feature data may be derived from a physiological signal associated with administration of a fluid bolus to a patient. For example, the physiological signal may be received prior to the feature data set. In some examples, the physiological signal is arterial blood pressure, a signal proportional to arterial blood pressure, or otherwise related to arterial blood pressure.

At a step 604, the process 600 involves receiving bolus log data. Bolus log data may be accessed from a log data store of a control device. In some examples, bolus log data may be provided to the data store from a pump controller and/or a sensor data analysis processor. Log data can include data related to a specific patient and/or data related to multiple patients.

At a step 606, the process 600 involves estimating a specific patient's response to a potential fluid bolus (e.g., IV fluid and/or medication). Estimating the patient response may involve evaluating and/or comparing the feature data and/or bolus log data. In some examples, estimation of patient response may be performed by a bolus recommendation engine as described herein. Estimating the patient's response may involve receiving and/or generating a recommendation indicating a predicted change in one or more physiological parameters of the patient in response to one or more fluid boluses.

At decision block 608, the engine may determine whether to suggest fluid based on the estimated patient response. The determined suggestion and/or recommendation may be presented to a clinician for consideration. However, in some examples, the suggestion determined at decision block 608 may not be presented to a clinician. Rather, subsequent suggestions determined at decision blocks 616 and/or 624 may be presented to clinicians. Where fluid is suggested, a first subset of features (e.g., the first subset 506 of FIG. 5) relating to the patient may be analyzed and/or applied to a responsiveness estimation engine 630 at a step 610. For example, the first subset may comprise a set of measurement types including the various features described herein. Features may include a sub-analysis of data tracked by one or more physiological sensors attached to and/or in communication with the patient. In some examples, the first subset may be a subset of the feature data set received at step 602. The responsiveness estimation engine 630 may be configured to evaluate and/or reevaluate suggestions made by the bolus recommendation engine 620.

Where fluid is not suggested, a negative response may be generated at a step 614 and/or a fluid bolus may not be provided. Where a test response is suggested (e.g., fluid is neither suggested nor not suggested), a second subset of features (e.g., the second subset 508 of FIG. 5) relating to the patient may be analyzed and/or applied to a responsiveness estimation engine 630 at a step 612. For example, the second subset may comprise a set of measurement types including the various features described herein. Features may include a sub-analysis of data tracked by one or more physiological sensors attached to and/or in communication with the patient. In some examples, the second subset may be a subset of the feature data set received at step 602.

The responsiveness estimation engine 630 may be configured to apply deep learning and/or machine learning to analyze the first subset and/or second subset of features to determine whether to recommend a fluid bolus (e.g., normal or full bolus) or to recommend a test bolus (e.g., test and/or partial bolus) at a decision block 616 and/or decision block 624.

Where a fluid bolus (e.g., full bolus) is recommended, a “recommend” response may be generated and/or outputted at a step 618 and/or step 626. Where a test bolus (e.g., test and/or partial bolus) is recommended, a “test” response may be generated and/or outputted at a step 622 and/or step 628. The analysis performed at decision blocks 616 and/or 624 may be identical and/or similar, however different subsets of features may be applied by the engine 630 at blocks 616 and 624.

The outputs at steps 618, 622, 626, and/or 628 may be provided to a display device (e.g., a monitor) and/or to a computer and/or robotic system configured to automatically dispense fluid in response to the outputs. For example, in response to outputting a “recommend” response, a “recommend” message and/or option may be presented to a display device and/or a fluid pump may automatically be activated to provide a bolus to the patient. In response to outputting a “test” response, a “test” message and/or option may be presented to a display device and/or a fluid pump may automatically be activated to provide a test bolus to the patient.

The engine 630 may be configured to provide a double check and/or second evaluation of the patient's responsiveness and/or anticipated responsiveness to a fluid bolus. In some examples, the engine 630 may confirm the outcome determined by the engine 620 and/or may modify the outcome determined by the engine 620 based on a deeper analysis and/or a more focused analysis of particular features of the feature data set received at step 602.

While the responsiveness estimation engine 630 is shown receiving recommendations from the bolus recommendation engine 620, in some examples, the responsiveness estimation 630 may be configured to determine recommendations without receiving a previous recommendation. For example, the engine 630 may be configured to generate fluid bolus recommendations using only feature data and/or bolus log data and/or not using a suggested fluid recommendation from the engine 620. The engine 630 may be configured to apply subsets of the feature data or the full feature data set.

One or more fluid boluses may be delivered and/or administered to the patient based at least in part on recommendations generated in the process 600. For example, a control device may be configured to generate signals to one or more fluid pumps to cause the one or more fluid pumps to administer one or more fluid boluses to the patient in response to one or more generated recommendations.

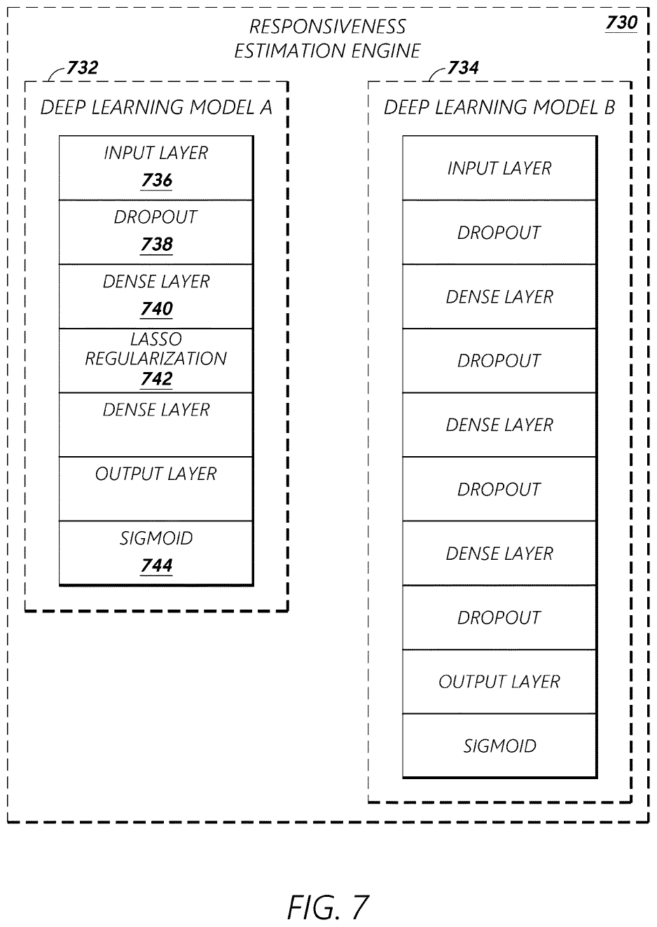

FIG. 7 illustrates an example structure of a responsiveness estimation engine 730 in accordance with one or more examples. In some examples, the engine 730 may be configured to apply machine learning and/or deep learning to feature data received at the engine 730. The engine 730 may comprise multiple models and/or data structures configured to analyze different sets and/or types of feature data. For example, the engine 730 may comprise a first deep learning model 732 and/or a second deep learning model 734. The engine 730 may utilize the first model 732 for a first subset and/or type of features and/or may utilize the second model 734 for a second subset and/or type of features. The first model 732 and/or second model 734 may be configured to receive an input of one or more features and/or sets of feature data and/or to output a “recommend” and/or “test” response, indicating a predicted responsiveness of a patient to a fluid bolus.

The first model 732 and/or second model 734 may comprise a neural network and/or one or more layers and/or functions configured to analyze feature data. In some cases, one or more layers and/or functions may be configured to remove unnecessary data and/or to apply weights and/or biases to one or more portions of data.

The first model 732 and/or second model 734 may comprise an input layer 736 configured to receive and/or process received feature data. In some examples, the input layer 736 may be configured to format the feature data into a format that is suitable for subsequent layers and/or functions of the first model 732 and/or second model 734.

In some examples, the first model 732 and/or second model 734 may comprise one or more dropout layers 738 configured to drop connections and/or prevent overfitting of the feature data. Dropout layers 738 may be employed following input layers 736 and/or dense layers 740. For example, a dropout layer 738 may remove some data added by a dense layer 740.

A dense layer 740 may comprise an architecture of neurons configured to apply weights to the feature data. An example dense layer 740 can connect neurons between layers of the first model 732 and/or second model 734. Dense layers 740 can detect complex patterns in the feature data and/or perform non-linear transformations (e.g., added weights and/or biases) to the feature data. The first model 732 and/or second model 734 can comprise multiple dense layers 740. In some examples, the second model 734 may comprise more dense layers 740 and/or more dropout layers 738 than the first model 732. For example, the second model 734 may be configured to analyze feature data where a “test” response was initially generated by an engine and/or feature data inputted to the second model 734 may have a higher degree of ambiguity and/or may comprise fewer connections relative to feature data inputted to the first model 732 (which may be configured to analyze feature data where a “recommend” response was initially generated by an engine). Accordingly, more dense layers 740 may be included in the second model 734 to focus the model on establishing connections between points of the feature data. The second model 734 may be relatively more complex and/or may comprise more layers relative to the first model 732.

The first model 732 may comprise a lasso regularization layer 742 configured to shrink (e.g., apply a reduced weight) to one or more features determined by the lasso regularization layer 742 to be relatively less important. In some cases, the layer 742 may be configured to encourage sparsity and/or drive some weights to zero to facilitate feature selection and/or simplify the first model 732.

In some examples, the first model 732 and/or second model 734 may comprise a sigmoid layer 744 configured to map the inputted and/or analyzed data to a value in a given range (e.g., between 0 and 1). The sigmoid layer 744 may be configured to create non-linearity in the feature data.

The engine 730 may comprise a neural network comprising coefficients derived from feature data input to the engine 730 from previous patients. Additionally, the engine 730 may be configured to generate additional coefficients dynamically from a current patient.

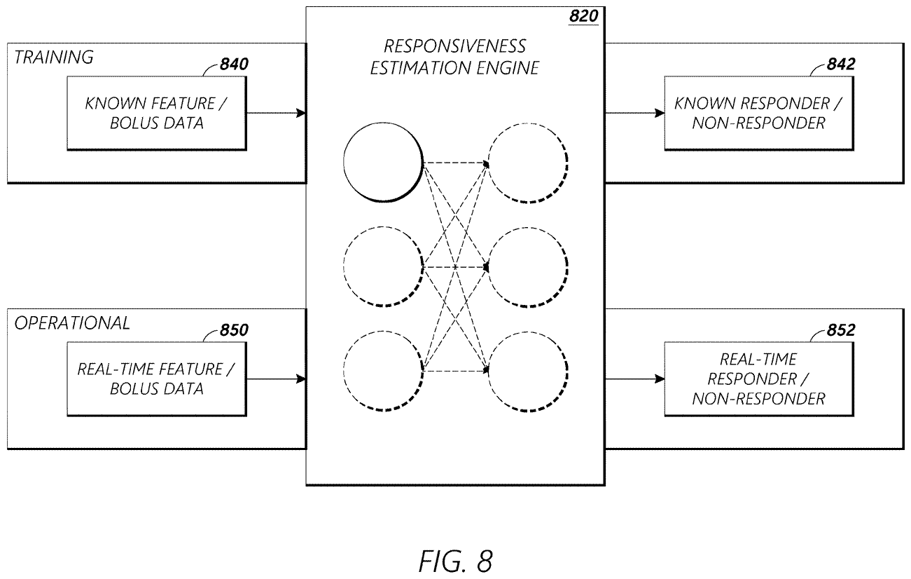

FIG. 8 illustrates a responsiveness framework 800 for predicting patient response to fluid boluses in accordance with one or more examples. The framework 800 may be embodied in certain control circuitry, including one or more processors, data storage devices, connectivity features, substrates, passive and/or active hardware circuit devices, chips/dies, and/or the like. For example, the framework 800 may be embodied in the responsiveness estimation engine 820 described in FIGS. 6 and 7. The framework 800 may be configured to employ machine learning functionality to perform automatic target detection on, for example, feature and/or bolus log data collected from one or more patients.

In some examples, the engine 820 may be trained according to known feature and/or bolus log data 840 and/or known patient response data 842. For example, the engine 820 may be provided feature data and/or log data previously collected from patients prior to receiving a fluid bolus. The engine 820 may also be provided with data relating to responsiveness of the patients following administration of the fluid bolus. For example, a match between input data for a previous patient and a known “recommend” (e.g., responder) or “test” (e.g., non- or partial-responder) may be provided to the engine 820 to train the engine 820 to match input data to a “recommend” or “test” outcome. The engine 820 may comprise any suitable or desirable transform and/or classification architecture, such as any suitable or desirable artificial neural network architecture.

The engine 820 may be configured to adaptively adjust one or more parameters or weights associated with training input/output data to correlate the known input and output data. For example, the engine 820 (e.g., multi-layer perception and/or convolutional neural network) may be trained using a labelled dataset and/or machine learning. In some implementations, the machine learning framework may be configured to execute the learning/training in any suitable or desirable manner.

The known response data 842 may be generated at least in part by manually labeling patient feature and/or log data with a “recommend” or “test” response. For example, manual labels may be determined and/or applied by a relevant medical expert to label where a patient may benefit from a fluid bolus. The known input/output pairs can indicate the parameters of the engine 820, which may be dynamically updatable in some embodiments.

The framework 800 may further be configured to generate real-time patient responder labels 852 associated with real-time feature and/or log data 850 using the trained version of the engine 820. For example, during a medical procedure, real-time feature and/or bolus log data 850 associated with a particular patient and/or one/or one or more previous patients may be processed using the engine 820 to generate real-time responder labels 852 (e.g., “recommend” or “test”) estimating the patient's response to an additional fluid bolus. In some examples, the engine 820 may be configured to weight and/or bias feature and/or log data provided to the engine 820 to correlate the patient-specific data to training input data and/or other input data previously analyzed at the engine 820.

The engine 820 can utilize any of a variety of machine learning methods, which can include logistic regression (LR), support vector machine (SVM), extreme gradient boosting (XGBoost), SVM+LR, and/or XGBoost+LR.

### ADDITIONAL EXAMPLES

Depending on the example, certain acts, events, or functions of any of the processes or algorithms described herein can be performed in a different sequence, may be added, merged, or left out altogether. Thus, in certain examples, not all described acts or events are necessary for the practice of the processes.

Example 1: A method for managing fluid administration to a patient, the method comprising: receiving a physiological signal; determining a plurality of features derived from the physiological signal associated with administration of a fluid bolus to a patient; determining a first fluid bolus recommendation indicating a predicted change in a physiological parameter of the patient in response to the fluid bolus based on the plurality of features; determining a second fluid bolus recommendation based on the first fluid bolus recommendation and a subset of the plurality of features; and providing the second fluid bolus recommendation.

Example 2: The method of any example herein, in particular example 1, wherein the physiological signal is arterial blood pressure or a signal proportional to arterial blood pressure.

Example 3: The method of any example herein, in particular example 1, further comprising generating the first fluid bolus recommendation based on the predicted change in the physiological parameter.

Example 4: The method of any example herein, in particular example 1, wherein the first fluid bolus recommendation comprises a recommendation of a normal bolus or a test bolus.

Example 5: The method of any example herein, in particular example 1, wherein the second fluid bolus recommendation is based on a first subset of the plurality of features.

Example 6: The method of any example herein, in particular example 5, wherein the second fluid bolus recommendation is based on a second subset of the plurality of features.

Example 7: The method of any example herein, in particular example 1, wherein the plurality of features comprises one or more of stroke volume, cardiac output, stroke volume variation, stroke volume index, and cardiac index of the patient.

Example 8: The method of any example herein, in particular example 1, wherein the plurality of features comprises one or more of rate of change of stroke volume, rate of change of cardiac output, rate of change of stroke volume variation, rate of change of stroke volume index, and rate of change of cardiac index.

Example 9: The method of any example herein, in particular example 1, wherein the second fluid bolus recommendation comprises a recommendation of a normal bolus or a test bolus.

Example 10: The method of any example herein, in particular example 1, wherein analyzing the subset involves adjusting one or more weights of the subset.

Example 11: The method of any example herein, in particular example 1, wherein analyzing the subset involves adjusting one or more biases of the subset.

Example 12: The method of any example herein, in particular example 1, further comprising accessing the plurality of features from one or more sensors attached to the patient.

Example 13: The method of any example herein, in particular example 1, further comprising providing the second fluid bolus recommendation to a display device.

Example 14: The method of any example herein, in particular example 1, further comprising providing the second fluid bolus recommendation to a pump system configured to automatically dispense the fluid bolus.

Example 15: A system comprising: one or more clinical sensors; control circuitry configured to: receive a physiological signal from the one or more clinical sensors; determine a plurality of features derived from the physiological signal associated with administration of a fluid bolus to a patient; determine a first fluid bolus recommendation indicating a predicted change in a physiological parameter of the patient in response to the fluid bolus based on the plurality of features; determine a second fluid bolus recommendation based on the first fluid bolus recommendation and a subset of the plurality of features; and provide the second fluid bolus recommendation.

Example 16: The system of any example herein, in particular example 15, wherein the physiological signal is arterial blood pressure or a signal proportional to arterial blood pressure.

Example 17: The system of any example herein, in particular example 15, wherein the control circuitry is further configured to generate the first fluid bolus recommendation based on the predicted change in the physiological parameter.

Example 18: The system of any example herein, in particular example 15, wherein the second fluid bolus recommendation is based on a first subset of the plurality of features.

Example 19: The system of any example herein, in particular example 18, wherein the second fluid bolus recommendation is based on a second subset of the plurality of features.

Example 20: The system of any example herein, in particular example 15, further comprising a display device, wherein the control circuitry is further configured to provide the second fluid bolus recommendation to the display device.

Example 21: A method for administering fluid to a patient, the method comprising: receiving a physiological signal; determining a plurality of features derived from the physiological signal associated with administration of a first fluid bolus to a patient; determining a first fluid bolus recommendation indicating a predicted change in a physiological parameter of the patient in response to the first fluid bolus based on the plurality of features; determining a second fluid bolus recommendation based on the first fluid bolus recommendation and a subset of the plurality of features; and delivering a second fluid bolus to the patient based at least in part on the second fluid bolus recommendation via one or more fluid pumps.

Example 22: The method of any example herein, in particular example 21, wherein the physiological signal is arterial blood pressure or a signal proportional to arterial blood pressure.

Example 23: The method of any example herein, in particular example 21, wherein the first fluid bolus recommendation comprises a recommendation of a normal bolus or a test bolus.

Example 24: The method of any example herein, in particular example 21, wherein the second fluid bolus recommendation is based on a first subset of the plurality of features.

Example 25: The method of any example herein, in particular example 24, wherein the second fluid bolus recommendation is based on a second subset of the plurality of features.

Example 26: The method of any example herein, in particular example 21, wherein the plurality of features comprises one or more of stroke volume, cardiac output, stroke volume variation, stroke volume index, cardiac index of the patient, rate of change of stroke volume, rate of change of cardiac output, rate of change of stroke volume variation, rate of change of stroke volume index, and rate of change of cardiac index.

Example 27: The method of any example herein, in particular example 21, wherein the second fluid bolus recommendation comprises a recommendation of a normal bolus or a test bolus.

Example 28: The method of any example herein, in particular example 21, wherein analyzing the subset involves adjusting one or more weights of the subset.

Example 29: The method of any example herein, in particular example 21, wherein analyzing the subset involves adjusting one or more biases of the subset.

Example 30: The method of any example herein, in particular example 21, further comprising accessing the plurality of features from one or more sensors attached to the patient.

Example 31: A system comprising: one or more clinical sensors; and control circuitry configured to: receive a physiological signal from the one or more clinical sensors; determine a plurality of features derived from the physiological signal associated with administration of a fluid bolus to a patient; determine a first fluid bolus recommendation indicating a predicted change in a physiological parameter of the patient in response to the fluid bolus based on the plurality of features; determine a second fluid bolus recommendation based on the first fluid bolus recommendation and a subset of the plurality of features; and deliver, via one or more fluid pumps, a second fluid bolus to the patient based at least in part on the second fluid bolus recommendation.

Example 32: The system of any example herein, in particular example 31, wherein the physiological signal is arterial blood pressure or a signal proportional to arterial blood pressure.

Example 33: The system of any example herein, in particular example 31, wherein the control circuitry is further configured to generate the first fluid bolus recommendation based on the predicted change in the physiological parameter.

Example 34: The system of any example herein, in particular example 31, wherein the second fluid bolus recommendation is based on a first subset of the plurality of features.

Example 35: The system of any example herein, in particular example 34, wherein the second fluid bolus recommendation is based on a second subset of the plurality of features.

Example 36: The system of any example herein, in particular example 31, wherein the plurality of features comprises one or more of stroke volume, cardiac output, stroke volume variation, stroke volume index, cardiac index of the patient, rate of change of stroke volume, rate of change of cardiac output, rate of change of stroke volume variation, rate of change of stroke volume index, and rate of change of cardiac index.

Example 37: The system of any example herein, in particular example 31, wherein the second fluid bolus recommendation comprises a recommendation of a normal bolus or a test bolus.

Example 38: The system of any example herein, in particular example 31, wherein determining the second fluid bolus recommendation based on the first fluid bolus recommendation and the subset of the plurality of features involves adjusting one or more weights of the subset of the plurality of features.

Example 39: The system of any example herein, in particular example 31, wherein determining the second fluid bolus recommendation based on the first fluid bolus recommendation and the subset of the plurality of features involves adjusting one or more biases of the subset of the plurality of features.

Example 40: The system of any example herein, in particular example 31, wherein the control circuitry is further configured to access the plurality of features from one or more sensors attached to the patient.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements, and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

1. A method for managing fluid administration to a patient, the method comprising:
receiving a physiological signal;
determining a plurality of features derived from the physiological signal associated with administration of a fluid bolus to a patient;
passing the plurality of features into a machine-learning model comprising a plurality of layers;
determining, based on passing the plurality of features into the machine-learning model, a first fluid bolus recommendation indicating a predicted change in a physiological parameter of the patient in response to the fluid bolus based on the plurality of features;
determining a second fluid bolus recommendation based on the first fluid bolus recommendation and a subset of the plurality of features; and
providing the second fluid bolus recommendation.

2. The method of claim 1, wherein the physiological signal is arterial blood pressure or a signal proportional to arterial blood pressure.

3. The method of claim 1, further comprising generating the first fluid bolus recommendation based on the predicted change in the physiological parameter.

4. The method of claim 1, wherein the first fluid bolus recommendation comprises a recommendation of a normal bolus or a test bolus.

5. The method of claim 1, wherein the second fluid bolus recommendation is based on a first subset of the plurality of features.

6. The method of claim 5, wherein the second fluid bolus recommendation is based on a second subset of the plurality of features.

7. The method of claim 1, wherein the plurality of features comprises one or more of stroke volume, cardiac output, stroke volume variation, stroke volume index, and cardiac index of the patient.

8. The method of claim 1, wherein the plurality of features comprises one or more of rate of change of stroke volume, rate of change of cardiac output, rate of change of stroke volume variation, rate of change of stroke volume index, and rate of change of cardiac index.

9. The method of claim 1, wherein the second fluid bolus recommendation comprises a recommendation of a normal bolus or a test bolus.

10. The method of claim 1, wherein determining the second fluid bolus recommendation based on the first fluid bolus recommendation and the subset of the plurality of features involves adjusting one or more weights of the subset of the plurality of features.

11. The method of claim 1, wherein determining the second fluid bolus recommendation based on the first fluid bolus recommendation and the subset of the plurality of features involves adjusting one or more biases of the subset of the plurality of features.

12. The method of claim 1, further comprising accessing the plurality of features from one or more sensors attached to the patient.

13. The method of claim 1, further comprising providing the second fluid bolus recommendation to a display device.

14. The method of claim 1, further comprising providing the second fluid bolus recommendation to a pump system configured to automatically dispense the fluid bolus.

15. A system comprising:
one or more clinical sensors; and
control circuitry configured to:
receive a physiological signal from the one or more clinical sensors;
determine a plurality of features derived from the physiological signal associated with administration of a fluid bolus to a patient;
pass the plurality of features into a machine-learning model comprising a plurality of layers;
determine, based on passing the plurality of features into the machine-learning model, a first fluid bolus recommendation indicating a predicted change in a physiological parameter of the patient in response to the fluid bolus based on the plurality of features;
determine a second fluid bolus recommendation based on the first fluid bolus recommendation and a subset of the plurality of features; and
provide the second fluid bolus recommendation.

16. The system of claim 15, wherein the physiological signal is arterial blood pressure or a signal proportional to arterial blood pressure.

17. The system of claim 15, wherein the control circuitry is further configured to generate the first fluid bolus recommendation based on the predicted change in the physiological parameter.

18. The system of claim 15, wherein the second fluid bolus recommendation is based on a first subset of the plurality of features.

19. The system of claim 18, wherein the second fluid bolus recommendation is based on a second subset of the plurality of features.

20. The system of claim 15, further comprising a display device, wherein the control circuitry is further configured to provide the second fluid bolus recommendation to the display device.

21. A method for administering fluid to a patient,
the method comprising: receiving a physiological signal;
determining a plurality of features derived from the physiological signal associated with administration of a first fluid bolus to a patient;
passing the plurality of features into a machine-learning model comprising a plurality of layers;
determining, based on passing the plurality of features into the machine-learning model, a first fluid bolus recommendation indicating a predicted change in a physiological parameter of the patient in response to the first fluid bolus based on the plurality of features;
determining a second fluid bolus recommendation based on the first fluid bolus recommendation and a subset of the plurality of features; and
delivering, via one or more fluid pumps, a second fluid bolus to the patient based at least in part on the second fluid bolus recommendation.

22. The method of claim 21, wherein the physiological signal is arterial blood pressure or a signal proportional to arterial blood pressure.

23. The method of claim 21, wherein the first fluid bolus recommendation comprises a recommendation of a normal bolus or a test bolus.

24. The method of claim 21, wherein the second fluid bolus recommendation is based on a first subset of the plurality of features.

25. The method of claim 24, wherein the second fluid bolus recommendation is based on a second subset of the plurality of features.

26. The method of claim 21, wherein the plurality of features comprises one or more of stroke volume, cardiac output, stroke volume variation, stroke volume index, cardiac index of the patient, rate of change of stroke volume, rate of change of cardiac output, rate of change of stroke volume variation, rate of change of stroke volume index, and rate of change of cardiac index.

27. The method of claim 21, wherein the second fluid bolus recommendation comprises a recommendation of a normal bolus or a test bolus.

28. The method of claim 21, wherein determining the second fluid bolus recommendation based on the first fluid bolus recommendation and the subset of the plurality of features involves adjusting one or more weights of the subset of the plurality of features.

29. The method of claim 21, wherein determining the second fluid bolus recommendation based on the first fluid bolus recommendation and the subset of the plurality of features involves adjusting one or more biases of the subset of the plurality of features.

30. The method of claim 21, further comprising accessing the plurality of features from one or more sensors attached to the patient.

31. A system comprising:
one or more clinical sensors; and
control circuitry configured to:
receive a physiological signal from the one or more clinical sensors;
determine a plurality of features derived from the physiological signal associated with administration of a fluid bolus to a patient;
pass the plurality of features into a machine-learning model comprising a plurality of layers;
determine, based on passing the plurality of features into the machine-learning model, a first fluid bolus recommendation indicating a predicted change in a physiological parameter of the patient in response to the fluid bolus based on the plurality of features;
determine a second fluid bolus recommendation based on the first fluid bolus recommendation and a subset of the plurality of features; and
deliver, via one or more fluid pumps, a second fluid bolus to the patient based at least in part on the second fluid bolus recommendation.

32. The system of claim 31, wherein the physiological signal is arterial blood pressure or a signal proportional to arterial blood pressure.

33. The system of claim 31, wherein the control circuitry is further configured to generate the first fluid bolus recommendation based on the predicted change in the physiological parameter.

34. The system of claim 31, wherein the second fluid bolus recommendation is based on a first subset of the plurality of features.

35. The system of claim 34, wherein the second fluid bolus recommendation is based on a second subset of the plurality of features.

36. The system of claim 31, wherein the plurality of features comprises one or more of stroke volume, cardiac output, stroke volume variation, stroke volume index, cardiac index of the patient, rate of change of stroke volume, rate of change of cardiac output, rate of change of stroke volume variation, rate of change of stroke volume index, and rate of change of cardiac index.

37. The system of claim 31, wherein the second fluid bolus recommendation comprises a recommendation of a normal bolus or a test bolus.

38. The system of claim 31, wherein determining the second fluid bolus recommendation based on the first fluid bolus recommendation and the subset of the plurality of features involves adjusting one or more weights of the subset of the plurality of features.

39. The system of claim 31, wherein determining the second fluid bolus recommendation based on the first fluid bolus recommendation and the subset of the plurality of features involves adjusting one or more biases of the subset of the plurality of features.

40. The system of claim 31, wherein the control circuitry is further configured to access the plurality of features from one or more sensors attached to the patient.
